(19) United States
(12) Patent Application Publication
(10) Pub. No.: US 2019/0254854 A1
(43) Pub. Date: Aug. 22, 2019

(54) WEARABLE ASSISTIVE DEVICE HAVING FOOT SUPPORT

(71) Applicant: LG ELECTRONICS INC., Seoul (KR)

(72) Inventors: Wonjun LEE, Seoul (KR); Bohyun NAM, Seoul (KR); Kyu Tae PARK, Seoul (KR); Jung Kyu SON, Seoul (KR); Seonil YU, Seoul (KR)

(21) Appl. No.: 16/274,798

(22) Filed: Feb. 13, 2019

Related U.S. Application Data
(60) Provisional application No. 62/730,399, filed on Sep. 12, 2018.
(60) Provisional application No. 62/730,400, filed on Sep. 12, 2018.
(60) Provisional application No. 62/730,412, filed on Sep. 12, 2018.
(60) Provisional application No. 62/730,420, filed on Sep. 12, 2018.

(30) Foreign Application Priority Data
Feb. 20, 2018 (KR) 10-2018-0019994
Mar. 14, 2018 (KR) 10-2018-0029842

(51) Int. Cl. A61F 5/01; A61H 1/02; B25J 9/00

(52) U.S. Cl. CPC A61F 5/0127; A61H 1/0266; B25J 9/0006; A61H 2201/1659; A61H 2201/165; A61H 2205/10; A61H 2201/5058; A61H 1/0262

(57) ABSTRACT

Disclosed herein is a wearable assistive device that may have a foot support capable of securing to shoes of various sizes. The foot support may include first and second supports that are slidably coupled to each other and adjusted in length, whereby a length of the foot support may be automatically adjusted in accordance with a shoe size of a user.

### CROSS-REFERENCE TO RELATED APPLICATION

This application claims priority under 35 U.S.C. to § 119 to U.S. Provisional Patent Application Nos. 62/730,399, 62/730,400, 62/730,412, and 62/730,420, all filed on Sep. 12, 2018, and also priority to Korean Application No. 10-2018-0019994, filed on Feb. 20, 2018 and Korean Patent Application No. 10-2018-0029842, filed on Mar. 14, 2018, whose entire disclosures are incorporated herein by reference.

### BACKGROUND

### 1. Field

This application relates to assistive and/or rehabilitative technology.

### 2. Background

In assistive and/or rehabilitative technology, an assistive device such as a wearable robot or a robotic exoskeleton may assist or augment a movement of a user. The wearable assistive device may have a multi-joint skeletal structure to assist a user's power or strength when a user wears the wearable assistive device. The user may wear the exoskeleton on an upper body, lower body, or an entire body. For example, for a lower body exoskeleton, the exoskeleton may assist with a walking, bending, or lifting action of the user by providing an assistive force generated from a driving apparatus, e.g., motor, to the user.

This wearable assistive device may be secured to a waist, a leg, and/or a foot or shoe of the user. In the wearable assistive device, a detachment mechanism which secures a body of the user may be an important factor that determines the convenience and efficiency of use.

KR Patent No. 10-1600600 (foot stretching device) and KR Patent No. 10-1363834 (assistance apparatus for movement of ankle joint) disclose ankle joint assisting apparatuses that secure the user's feet during use of a power assisting apparatus. A conventional ankle joint assisting apparatus will be described with reference to the same.

For reference, FIG. 1 is a view shown in KR Patent No. 10-1600600, and FIG. 2 is a view shown in KR Patent No. 10-1363834. Referring to FIG. 1, the ankle joint assisting apparatus may include a frame 10 to support a foot and an ankle of the user and a fixing member or strap assembly 30 to secure the foot.

The frame 10 may be divided into an ankle frame 11, a foot frame 13, and a toe frame 15 based on positions corresponding to an ankle, foot, and toe, respectively. The foot frame 13 may be provided with a length adjustment section 20 having a plurality of holes or slots formed on an inner side thereof and a length adjusting pin or screw 19 to adjust a length of the foot frame 13 in accordance with a shoe size of the user. There is an inconvenience in that the user may have to manually change a position of the length adjusting pin 19 in order to adjust the length of the foot frame. Since the length adjusting pin 19 is fastened to a hole or slot formed in an inner side of the length adjustment section 20, it may be adjustable only by a restricted or predetermined length, making it difficult to adjust the foot frame 13 to fit the user's foot exactly.

Further, the strap assembly 30 of the conventional ankle joint assisting apparatus may include an ankle strap 31 to secure the user's ankle, a top strap 33 to secure the top of the foot, and a toe strap 35 to secure the toe. The strap assembly 30 may secure the user's foot using Velcro.

Since lengths of the ankle, toe, and top straps 31, 33, and 35 may be predetermined, it may not be possible to adjust the lengths, i.e., tightness, of the strap assembly 30 in accordance with the user's foot size. The strap assembly 30 may thus have to be manufactured in accordance with the foot size of a particular user, decreasing versatility of the ankle assisting apparatus and increasing manufacturing costs.

Since the strap assembly 30 may be manufactured to fit a particular user's foot size, the user may have to choose between customizing the strap assembly 30 to fit a particular shoe or a barefoot, but the strap assembly 30 likely cannot accommodate both. Thus, a user may not be able to switch between shoes or choose to take off his shoes to use the ankle assisting apparatus.

The conventional ankle joint assisting apparatus shown in FIG. 2 may be provided with first and second supports 41 and 42 that are adjustably coupled to each other. The first and second supports 41 and 42 may include a front sole cap or toe cap 41 and a foot side frame or ankle support 42 that are slidably coupled to each other so that a length between the toe cap 41 and the ankle support 42 may be adjusted, and an elastic member or midsole support 44 to supply an elastic force between the toe cap 41 and the ankle support 42.

The user may secure his foot bare foot or a particular shoe to the ankle joint assisting apparatus. The toe cap 41 may be manufactured to cover a front of the foot, which may be inserted into in an inner space of the toe cap 41. However, since the toe cap 41 may be manufactured to fit a maximum foot size so that users having various sizes can use it, there is a problem in that a user having a relatively small foot may not be secured properly in the inner space of the toe cap 41. In addition, the foot may be easily detached from the ankle joint assisting apparatus while walking since a fixing member or strap for is omitted.

Further, in the ankle joint assisting apparatus of FIG. 2, when a forward force is applied to the toe cap while walking, the elastic member 44 may be stretched and so a length of the elastic member 44 may be increased, which may loosen the ankle joint assisting apparatus so that it is no longer secured.

The above references are incorporated by reference herein where appropriate for appropriate teachings of additional or alternative details, features and/or technical background.

### BRIEF DESCRIPTION OF THE DRAWINGS

Embodiments will be described in detail with reference to the following drawings in which like reference numerals refer to like elements, and wherein:

FIGS. 1 and 2 are views explaining an ankle joint assisting apparatus according to related art;

FIG. 3 is a perspective view showing a wearable assistive device according to an exemplary embodiment;

FIG. 4 is a side view showing the wearable assistive device of FIG. 3;

FIG. 5 is a perspective view showing a foot support of a wearable assistive device according to an exemplary embodiment;

FIG. 6 is one side view showing the foot support of FIG. 5;

FIG. 7 is the other side view showing the foot support of FIG. 5;

FIG. 8 is a plan view showing the foot support of FIG. 5;

FIG. 9 is a rear view showing the foot support of FIG. 5;

FIG. 10 is a front view showing the foot support of FIG. 5;

FIG. 11 is a cross-sectional view taken along line A-k of FIG. 10;

FIG. 12 is an exploded perspective view showing the foot support of FIG. 5;

FIG. 13 is an exploded perspective view showing the first support of FIG. 12;

FIG. 14 is an exploded perspective view showing the second support of FIG. 12;

FIG. 15 is an exploded perspective view showing the strap of FIG. 12;

FIG. 16 is an exploded perspective view showing the button dial of FIG. 12;

FIG. 17 is a view for explaining a movement between the first support and the second support of the foot support of FIG. 5;

FIGS. 18 and 19 are views explaining operations of the strap and the button dial portion provided in the foot support of FIG. 5;

FIG. 20 is a view for explaining an operation of the foot support of FIG. 5; and

FIG. 21 is a view for explaining a donning process of a user with respect to the foot support of FIG. 5.

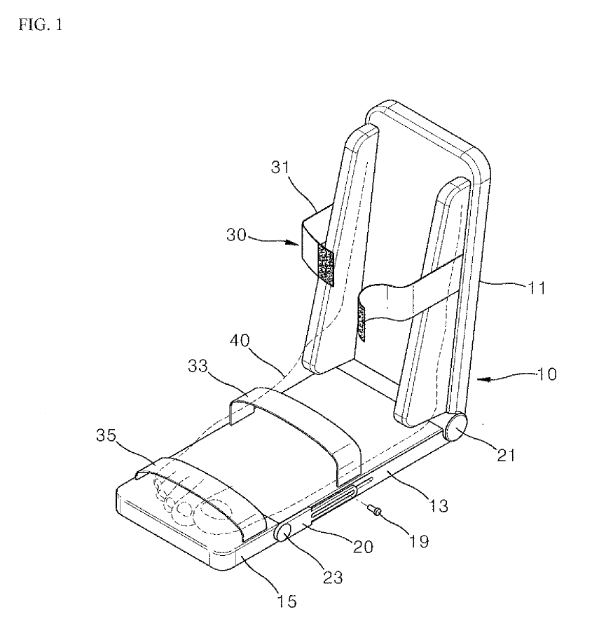

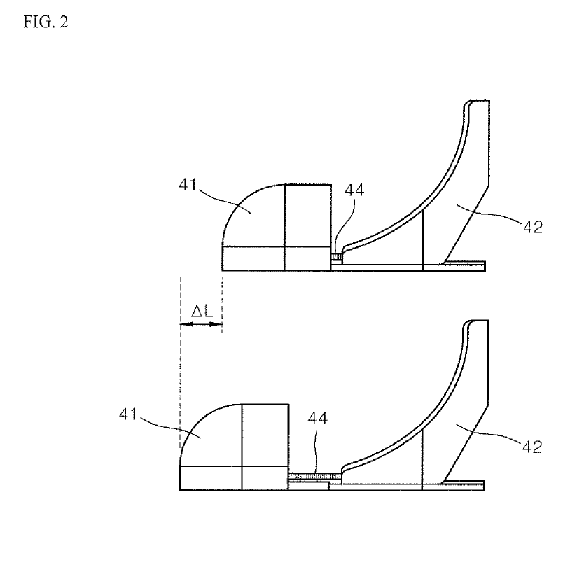

### DETAILED DESCRIPTION

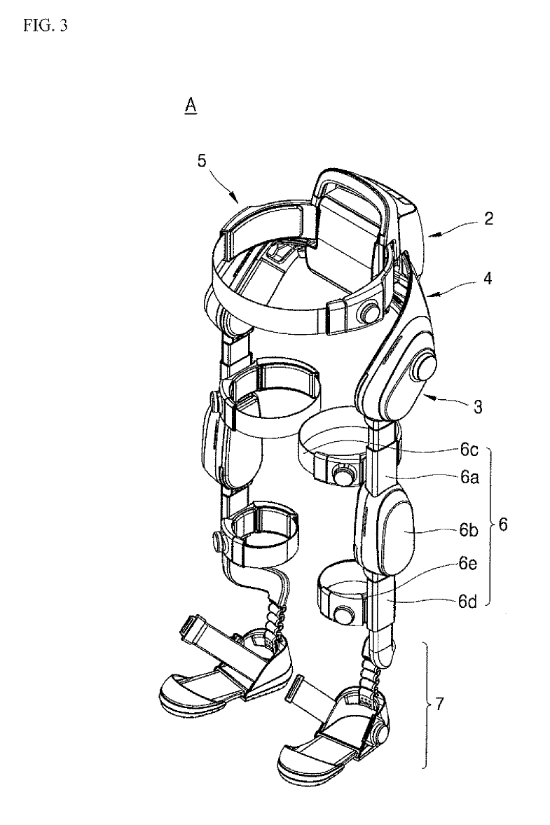

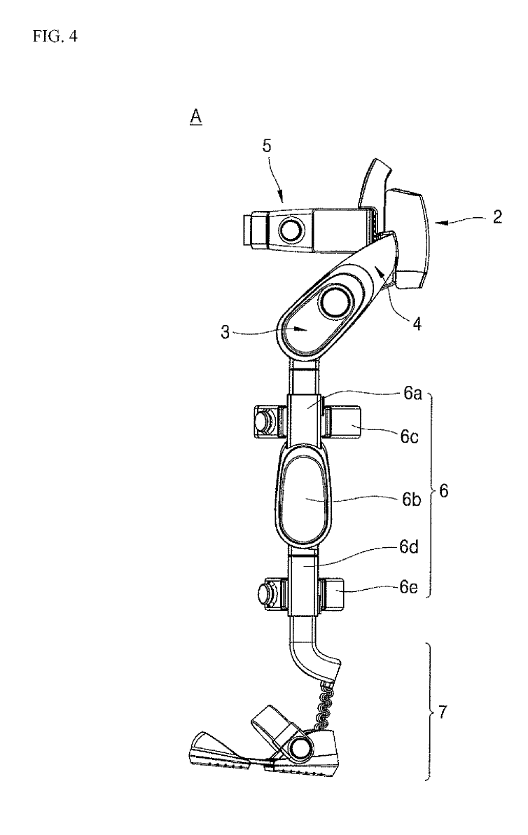

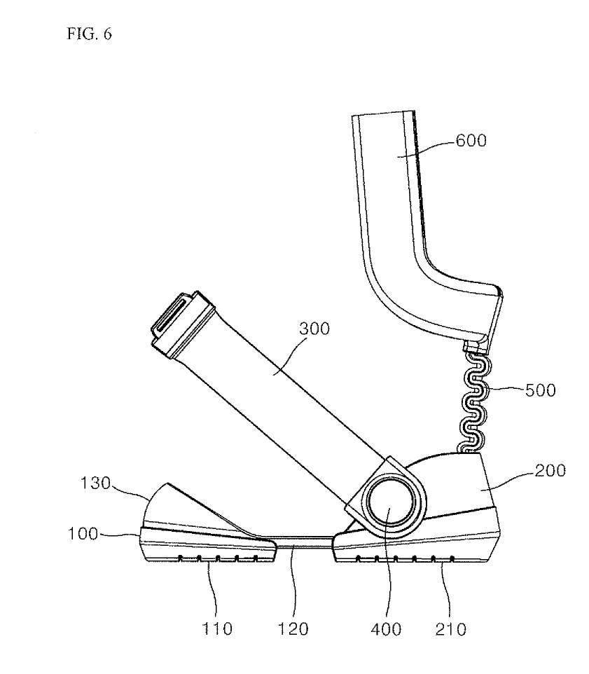

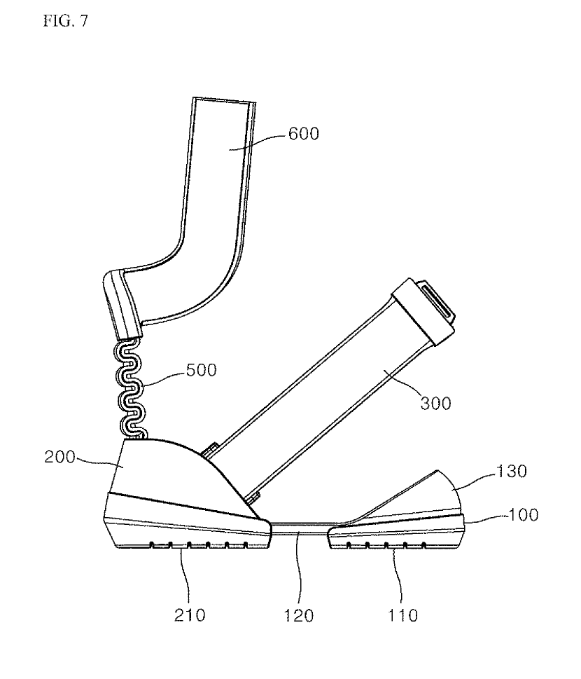

Hereinafter, a foot support included in a wearable assistive device such as a wearable robot, e.g., exoskeleton, according to an exemplary embodiment will be described with reference to FIGS. 3 to 21. Referring to FIGS. 3 and 4, a wearable assistive device such as a wearable robot A, and more specifically an exoskeleton, may be worn on a lower body of the user, and may assist a lower body of the user.

The exoskeleton A may mainly include a main controller housed in a lumbar/back frame 2 and a subcontroller housed in an actuated hip joint 3 for function control, a main frame 4 mounted on or secured to a pelvis or hips, a subframe or waist/pelvic frame 5 secured to a pelvis and/or a waist, a leg or leg assembly 6 that is secured to a leg of the user, and a foot support or assembly 7 that secures to a foot or shoe.

Specifically, the lumbar/back frame 2 may be provided on the main frame 4 and positioned on a lower back, e.g., lumbar, of the user. The main controller in the lumbar/back frame 2 may adjust a width of the main frame 4 to accommodate various body sizes. In addition, the lumbar/back frame 2 may include a battery pack that supplies a power source to operate the exoskeleton A. The subframe 5 may be provided on a front side of the lumbar/back frame 2. Details of the main controller and the subcontroller can be found in U.S. application Ser. Nos. ______ (Attorney Docket No. DAE-0073) filed on ______ and ______ (Attorney Docket No. DAE-0074) filed on ______, the entire disclosures of which are incorporated by reference herein.

The subframe 5 may support the waist of the user and may be secured to the waist via a belt whose length may be adjusted in one-touch dial manner. The subframe 5 may include an impact portion having a shock-absorbent material that contacts the waist of the user to improve comfort. Details of the subframe 5 may be found in U.S. application Ser. Nos. ______ (Attorney Docket No. DAE-0076) filed on ______, ______ (Attorney Docket No. DAE-0077) filed on ______, and ______ (Attorney Docket No. DAE-0086) filed on ______, the entire disclosures of which are incorporated by reference herein.

The main frame 4 may support the lumbar/back frame 2 and may have a shape configured to cover the hips or pelvis, e.g., ilium, of the user from a first side, e.g., left side, of the user to a second side, e.g., right side. The main frame 4 may be formed to have a substantially “U”-shape, and a bent portion or center of the main frame 4 may be positioned at the rear of the user. The lumbar/back frame 2 may be mounted on the center of the main frame 4. However, this is merely one example, and a position of the lumbar/back frame 2 may be modified in various ways. Opposite ends of the main frame 4 may extend downward along the hips or ilium of the user. The subcontroller may be provided at or above the ends in inclined portions or inclined extensions of the main frame 4.

The subcontroller may adjust a strength or magnitude of an assistive force that assists a power or strength of the user at a hip joint. The subcontroller may adjust the assistive force in a rotary dial manner. The subcontroller and/or main frame 4 may be provided with an indicator to indicate a magnitude of the assistive force with a lamp, light, or light-emitting device such as a light-emitting diode (LED). A driving means e.g., a motor or actuator (electric, hydraulic, or pneumatic) to supply the assistive force may be provided in the actuated hip joint 3. A leg assembly 6 may be coupled to a lower side of the actuated hip joint 3.

There may be a pair of leg assemblies 6, which may be worn on both legs of the user, respectively. Each leg assembly 6 may include an upper leg frame 6a that is secured to a thigh of the user via a leg belt 6c, an actuated joint 6b to supply an assistive force, and a lower leg frame 6d that is secured to a calf via a leg belt 6e. The leg belts 6c and 6e may be adjustable in a one-touch dial manner. Details of the leg belts 6c and 6e may be found in U.S. application Ser. Nos. ______ (Attorney Docket No. DAE-0084) filed on ______, ______ (Attorney Docket No. DAE-0085) filed on ______, and ______ (Attorney Docket No. DAE-0086) filed on ______, the entire disclosures of which are incorporated by reference herein.

The upper leg frame 6a, the actuated joint 6b, and the lower leg frame 6d may be provided at sides of the user's leg so as not to interfere with a movement of the user when he bends his joints. The upper leg frame 6a and the lower leg frame 6d may rotate in a direction corresponding to a movement of a knee joint of the user via the actuated joint 6b. The actuated joint 6b may be provided between the upper and lower leg frames 6a and 6d, and may include a motor and a gear set or actuator (e.g., electric, hydraulic, or pneumatic). The motor and the gear set may generate an assistive force that assists the power or strength of the user at a knee joint. For reference, various constituent elements capable of generating an assistive force may be used for the actuated joint 6b and the actuated hip joint 3. The actuated joint 6b and the actuated hip joint 3 may allow the user to bend his thigh or hip (at the actuated hip joint 3) or his calf (at the actuated joint 6b). The user may thus rotate his hip and knee joints in a sagittal plane of motion.

The upper leg frame 6a may be extended outward, e.g., to the left or right, in a frontal plane of motion by a predetermined angle by a hip joint structure (not shown) of the main frame 4. Further, the upper leg frame 6a and the lower leg frame 6d may have multi-joint structures capable of adjusting an inclination inward or outward to accommodate a natural inclination or leg shape of the user. The upper and lower leg frames 6a and 6d may also have multi-frame structures that allow an adjustment in length. Details of the main frame 4 and the upper and lower leg frames 6a and 6d can be found in U.S. application Ser. No. ______ (Attorney Docket No. DAE-0075), the entire contents of which is incorporated herein by reference.

The foot support 7 to secure the foot or the shoe of the user may be provided at a lower end of the lower leg frame 6d. The foot support 7 may secure to not only a barefoot of the user but also a sock or shoe of the user. Hereinafter, an example where a shoe of the user is secured to the foot support 7 will be described for ease of explanation.

The foot support 7 may be coupled to a lower end of the leg assembly 6 and support the shoe of the user. The foot support 7 may have an adjustable length where the shoe of the user is inserted, which is described in detail later. Further, the foot support 7 may be provided with a strap or belt described in detail later to cover and secure an upper surface of the shoe of the user. The strap may be adjustable Therefore, the foot support 7 may secure shoes of various users regardless of a type and a size of the shoe. The foot support 7 may be formed to have a shape corresponding to the shoe of the user, and may include a structure for simple fastening and unfastening of the shoe. For example, the foot support 7 may have a sandal structure. However, such a structure of the foot support 7 is merely an example, and various methods capable of securing the shoe may be applied.

The exoskeleton A is not limited to a lower body exoskeleton. The foot support 7 may be optional, and the exoskeleton A may be an upper body exoskeleton, lower body exoskeleton, or an exoskeleton configured to be supported on an entire body of a user. For example, exoskeleton A may instead be an upper body exoskeleton, which may have a lumbar/back support 2 housing a main controller, a main frame 4, a subframe 5, a limb assembly 6 extending upward from the main frame 4, and at least one actuated joint 3 or 6b. An exoskeleton A may also fit onto a complete or entire body of the user to accommodate both leg and arm limbs.

Although not shown, embodiments disclosed herein may not be limited to a complete lower body exoskeleton based on an intended use of the exoskeleton. For example, the actuated joint 6b, the lower leg frame 6d, the leg belt 6e, and the foot support 7 may be omitted. Thus, an exoskeleton A may provide assistance to the user at the actuated hip joint 3, and may include a main frame 4, a lumbar/back frame 2, and a leg assembly 6 that includes only an upper leg frame 6a that secures to a thigh of the user via a leg belt 6c. Such an upper leg exoskeleton A may have a hip structure substantially the same as or similar variations to the hip structure of the main frame 4 described in detail herein.

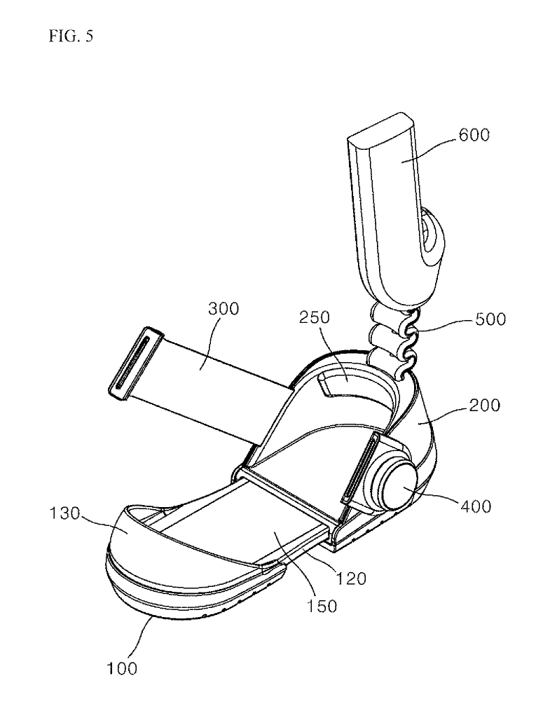

A detailed description of a structure and an operation of the foot support 7 will be described below with reference to FIGS. 5 to 21. Referring to FIGS. 5 to 10, the foot support 7 according to an exemplary embodiment may include first, e.g., front, and second, e.g., rear, supports 100 and 200 constituting a foot support structure, a binding belt or strap 300, a button dial or knob 400, an ankle support or ankle assembly 500, and a connection frame 600. The first and second supports 100 and 200 may form a foot supporting structure.

The first and second supports 100 and 200 may partially cover the user's shoe. For example, the first support 100 may cover the user's toes, and the second support 200 may cover the user's heel. The first support 100 and the second support 200 may be slidably coupled to each other.

The above-described first and second supports 100 and 200 are merely one exemplary embodiment, and the shoe may be fixed by other structures. Any structure may be applied to the first and second supports 100 and 200 to secure and support the foot of the user.

The first support 100 may have a structure to support a front of the shoe. The first support 100 may be formed to partially cover the front of the shoe near or over the toes. The first support 100 may have a curved semi-spherical or semi-ellipsoid shape. The first support 100 may be rounded to surround a front section of the shoe of the user.

The first support 100 may include a first outer sole 110 that touches to a ground or floor surface, a midsole or a shank 120 to be inserted into the second support 200, and a front support or toe cover 130 that extends upward from a first end of the midsole 120.

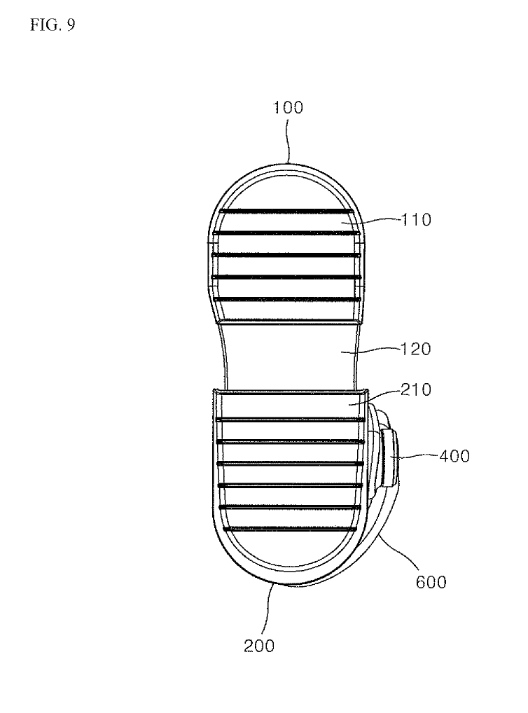

The first outer sole 110 may be provided under or below the midsole 120. The first outer sole 110 may grip the surface of the floor and increase a friction between the foot support 7 and the floor. Various patterns capable of enhancing a frictional force with the ground may be formed on the bottom surface of the first outer sole 110. For example, FIG. 9 shows a linear pattern, but, this disclosure is not limited thereto, and the first outer sole 110 may be formed with various patterns such as a diagonal line, a zigzag pattern, concave-convex protrusions, etc.

The midsole 120 may have a long and flat shape to support the front of the shoe of the user. The midsole 120 may include a section that is inserted into an inner space or receiving space 235 of the second support 200 such that the first support 100 and the second support 200 are slideably connected. The second support 200 may also have an inner sole (not shown). The entire length of the foot support 7 may be adjusted based on how far the midsole 120 is inserted into the inner space 235 of the second support 200. Since the first outer sole 110 is provided below the midsole 120, a central section of the midsole 120 may be spaced apart from the ground.

The toe cover 130 may have a shape to partially cover a front of the shoe to prevent the foot support 7 from falling or sliding off of the foot. The toe cover 130 may extend upward and may have a curved semi-spherical or semi-ellipsoid shape that partially covers toes or a front portion of the shoe of the user. The toe cover 130 may also be referred to as a toe box or toe cap. The toe cover 130 may be create an acute angle with the midsole 120, thereby partially covering a front of the shoe, while most of the upper surface of the show is exposed.

The second support 200 may have a shape to support a rear of the shoe. The second support 200 may have an inner space 235 in which the outer sole 120 of the first support 100 is inserted and coupled to a supporting surface to support the shoe, thereby adjusting a length between the first support 100 and the second support 200.

A lower surface of the second support 200 may be formed with a second outer sole 210, which may contact he ground. The second outer sole 210 may have various patterns capable of enhancing a frictional force with the ground.

Like the first outer sole 110, a linear pattern is shown on the second outer sole 210 in FIG. 9. However, embodiments are not limited thereto, and the second outer sole 210 may have various patterns such as a diagonal line, a zigzag pattern, and various concave recesses and/or convex protrusions.

A heel cover or heel cushion 250 may be provided on an inner side of the second support 200 to prevent the rear of the shoe from slipping from the second support 200. The heel cushion 250 may be formed to protrude outward from an inner surface of the second support 200 and may be provided on an upper side of the second support 200. The heel cushion 250 may support an upper heel (e.g., a lower Achilles tendon) of the user. The heel cushion 250 may be made of a material having elasticity or a buffering force or resilience. Accordingly, when the user who wears the exoskeleton A walks, bends, or lifts, the heel cushion 250 may alleviate an impact transferred to the user's heel and improve a comfort.

In an alternative embodiment, the first support 100 and the second support 200 may be integrally formed in the same process. For example, the first support 100 and the second support 200 may support a lower surface of the shoe of the user as an integral structure rather than as two supports slideably connected. The first and second supports 100 and 200 may be formed in a flat panel structure to support the shoe. However, this is merely one example, and the first and second supports 100 and 200 may be formed in various shapes capable of supporting the shoe.

The strap 300 may extend from a first side of the second support 200 to be coupled to a knob or button dial 400 provided on a second side of the second support 200. The strap 300 may be fastened to the button dial 400 to secure a top of the shoe of the user to the foot support 7. The strap 300 and the button dial 400 may be implemented as a fastening or buckling mechanism. The strap 300 may secure the user's shoe to the foot support 7 to be in close contact therewith, and may reduce an impact applied to the user's foot, thereby improving a comfort of the foot support 7.

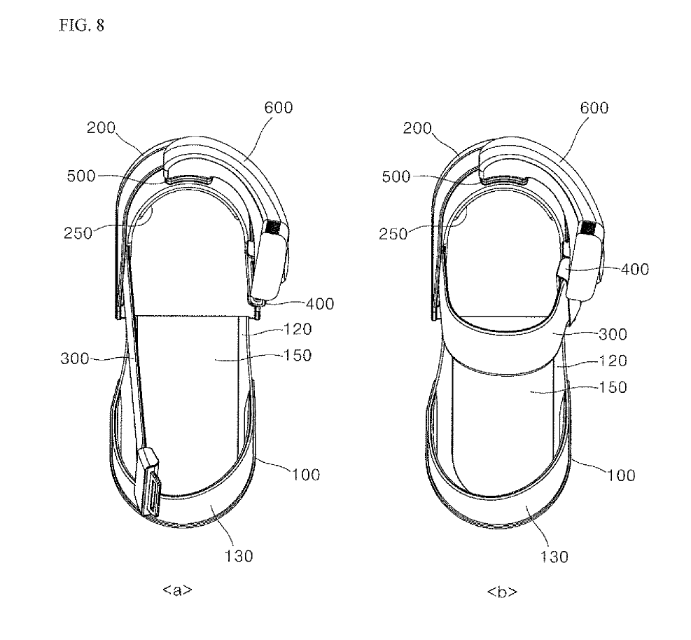

Referring to FIG. 8, FIG. 8(a) shows the foot support 7 before fastening the strap 300 to the button dial 400, and FIG. 8(b) shows the foot support 7 after fastening the strap 300 and the button dial 400. The strap 300 may be partially inserted into the second support 200 via the button dial 400, and may have a length which may be adjusted in accordance with a shoe size of the user. As a result, the strap 300 may prevent the user's shoe from coming off of the foot support 7.

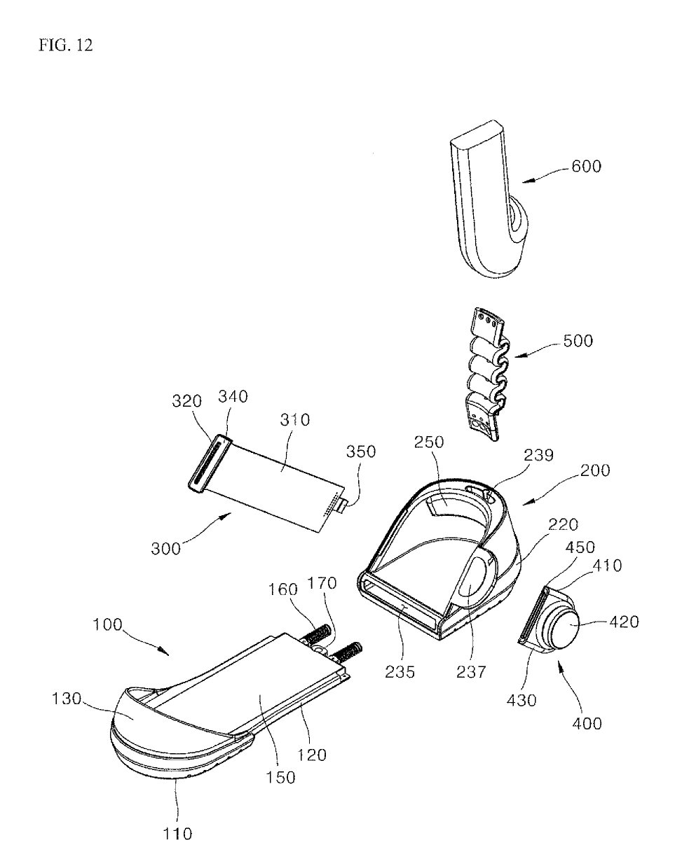

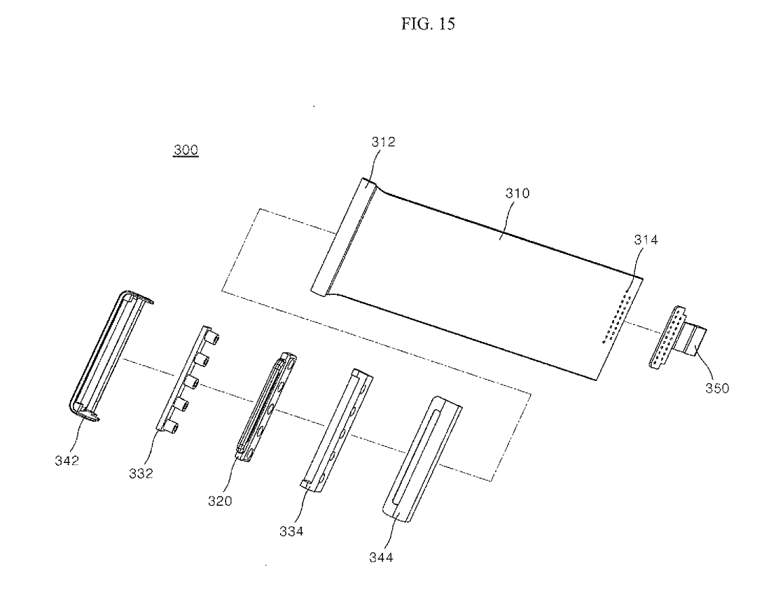

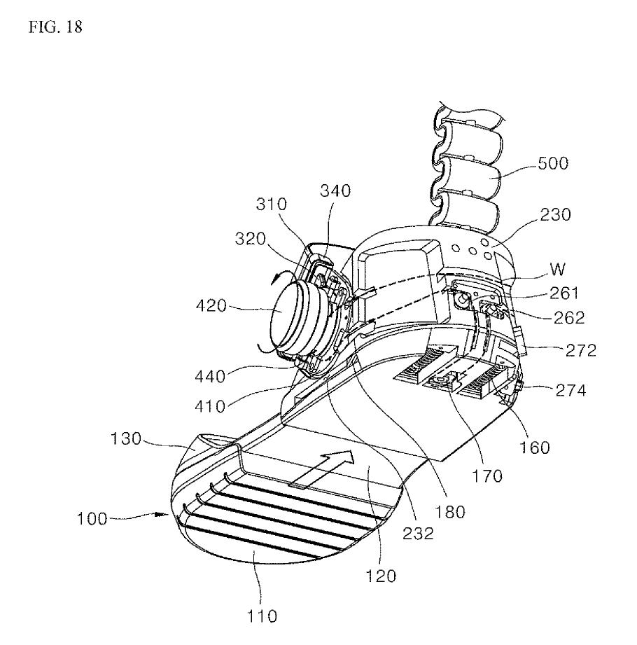

Referring to FIG. 12, the strap 300 may include a band 310, a binding clip or buckle 320 that is installed to one end of the band 310 to be coupled to the button dial 400, a buckle cover or buckle base 340 that covers where the buckle 320 and the band 310 connect, and a wire guide or wire connector 350 that is installed at a second end of the band 310 to be coupled to a wire (W of FIG. 18). The buckle 320 may also be referred to as a buckle tongue or latch, and details of the buckle 320 and the buckle base 340 will be described later with reference to FIG. 15. Details of the wire W will be described later with reference to FIG. 18.

The band 310 may be made of or include an elastic or flexible material. For example, the band 310 may be manufactured by weaving a number of elastic or flexible fibers. However, this is merely one example, and the band 310 may be made of materials having various elasticity or flexibility.

The button dial 400 may be provided on the second side of the second support 200 and coupled to the strap 300 to adjust an exposed or outside length of the strap 300. The button dial 400 may have a rotatable dial shape protruding from the second support 200. The button dial 400 may fasten or separate the strap 300 to or from the button dial 400 in a one-touch dial manner, or adjust the length of the strap 300.

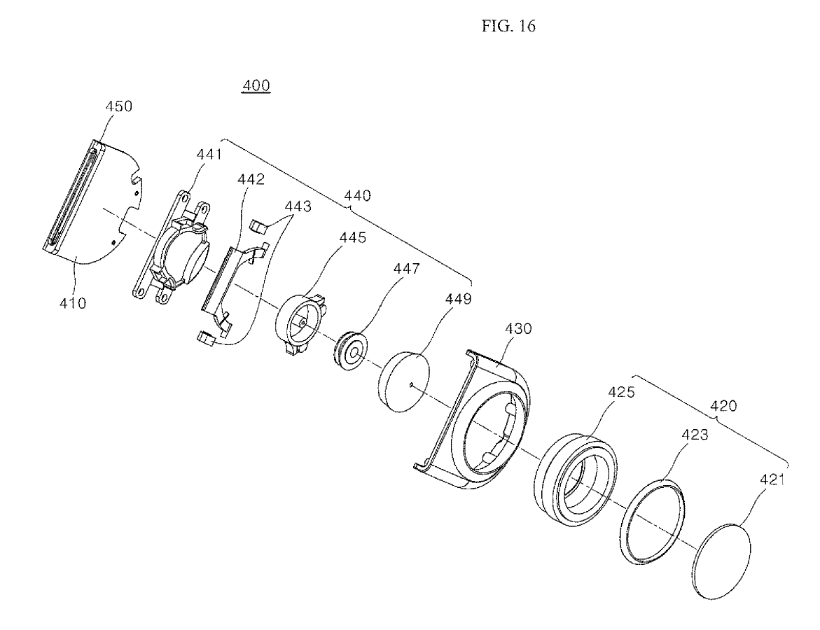

For example, when a dial of the button dial 400 is rotated when the strap 300 is fastened to the button dial 400, the length of the strap 300 may be adjusted, and the strap 300 may thus be tightened or loosened. Further, when the dial of the button dial 400 is pulled outward, the strap 300 may be unfastened from the button dial 400. However, this is merely one example, and the button dial 400 may be operated in various ways. Details of the button dial 400 will be described later with reference to FIG. 16.

The ankle support 500 may be provided to cover or extend behind the Achilles tendon of the user. The ankle support 500 may have a bottom end fixed to a center of an upper surface of the second support 200, and a top end coupled to the connecting frame 600. A central axis of the ankle support 500 may be provided in parallel to a center axis of the ankle of the user. The ankle support 500 may have a winding or accordion shape having a predetermined width. For example, the ankle support 500 may have a cross-section in which a “ [symbol] ”-shape is repeated or a cross-section having a zigzag shape.

When viewed from the front of the foot support 7, the ankle support 500 may be formed to have a predetermined width. However, this application is not limited thereto, and the ankle support 500 may be formed to have various cross-sections and widths. Due to these shapes, the ankle support 500 may be stretched and contracted within a predetermined range.

The ankle support 500 may be made of an elastic material such as any one of or a combination of two or more of a natural rubber, EPDM, polybutadiene, a nitrile rubber, a synthetic rubber, polyurethane and a silicone rubber. Such an elastic material may be manufactured by a wet spinning, a dry spinning, a melt spinning, and a polymerization reaction spinning method, etc. Also, the elastic material may be manufactured by mixing with a fabric or fiber having flexibility. However, this is merely one example, and a type and a manufacturing method of the elastic material are not limited thereto.

The ankle support 500 may allow the foot support 7 to move or rotate in forward, backward, leftward, rightward, upward, and downward directions based on the connecting frame 600 in response to an ankle movement of the user, and may expand a movable range of the foot support 7. At this time, the ankle support 500 may have a three-dimensional rotation relative to one point of action where the ankle support 500 connects to the connecting frame 600.

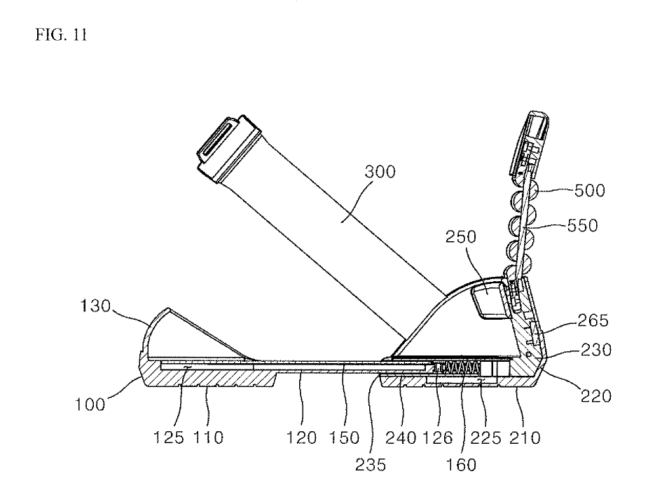

As a result, the ankle support 500 may support the ankle so as to allow a complicated or natural ankle movement of the user. Such a structure of the ankle support 500 may allow the user to easily change directions, thereby increasing a degree of freedom of a movement of the user. Details of the ankle support 500 are found in U.S. application Ser. No. ______ (Attorney Docket No. DAE-0072) filed on ______, the entire contents of which is incorporated by reference herein. A coupling of the ankle support 500 to the second support 200 will be described in more detail later with reference to FIG. 11.

The connecting frame 600 may connect the ankle support 500 and the leg assembly 6, as shown in the drawings, but embodiments disclosed herein are not limited thereto. In an alternative embodiment, the connecting frame 600 may be omitted, and the ankle support 500 may be directly connected to the lower end of the leg assembly 6.

The connecting frame 600 may extend downward from a side of the leg of the user so as not to interfere with an ankle bone of the user during walking, bending, or lifting, and the connecting frame 600 may curve above the ankle bone to extend to a rear of the leg of the user. Subsequently, the connecting frame 600 may couple to a rear of the ankle support 500.

A first side of the connecting frame 600 may extend in a longitudinal direction of the ankle support 500 behind an upper Achilles tendon of the user, and the a second side of the connecting frame 600 may bend or curve from the first side to extend to a side surface of the leg of the user. As a result, the connecting frame 600 may minimize an interference between a the ankle bone and the exoskeleton A during walking, bending, or lifting. The connecting frame portion 600 may further minimize physical impact between the leg of the user and the exoskeleton A, and prevent the body of the user from being burdened or fatigued. The connecting frame 600 may this improve a comfort and convenience. Further, the user may use the exoskeleton A for a long time, improving a length of assistance or treatment.

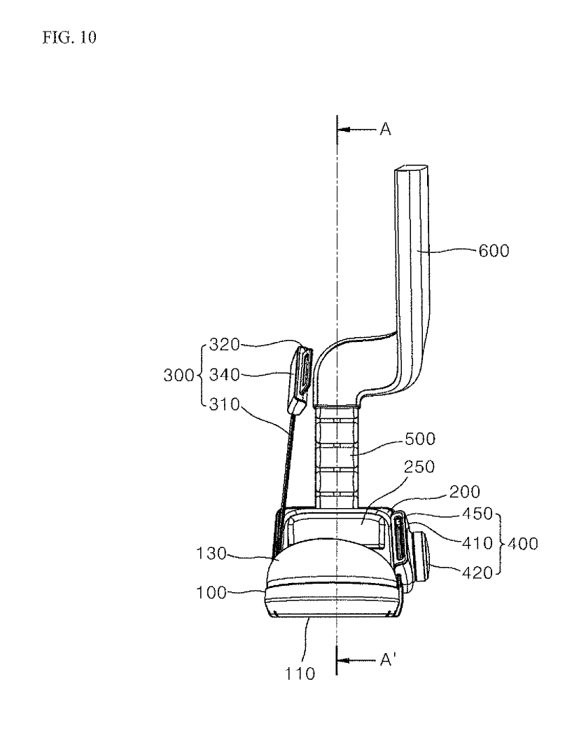

Hereinafter, a coupling of the ankle support 500 will be described in detail with reference to FIG. 11. FIG. 11 is a cross-sectional view taken along line A-A′ of FIG. 10. Referring to FIG. 11, a portion of the midsole 120 may be inserted into an inner space or receiving space 235 of the second support 200. The midsole 120 may be coupled to slide in the inner space 235 of the second support 200.

As will be described later in detail, the second support 200 may include a rear housing 220 that forms an outer surface of the second support 200 and a rear body 230 that is coupled to an inner surface of the rear housing 220. The inner space 235 may extend into the rear body 230, and, although not shown, may also extend in the rear housing 220. An elastic member 160 to supply an elastic force may be installed in the inner space 235 between the midsole 120 and the rear body 230. The elastic member 160 may be a spring, (e.g., coil spring) as shown in the drawings. Alternatively, the elastic member 160 may be a rubber band or other type of spring to supply an elastic force.

The elastic member 160 may have a first end that is fixed to a protruding portion or protrusion 126 formed at a first end of the midsole 120. A second end of the elastic member 160 may be fixed inside the inner space 235. This configuration may allow the elastic member 160 to supply an elastic or restoring force between the first support 100 and the second support 200.

For example, when a distance between the first support 100 and the second support 200 is greater than a predetermined distance, the elastic member 160 may stretch and expand, and may apply a force such that the distance between first support 100 and the second support 200 may be reduced. When the user pushes the first support 100 forward during walking, bending, or lifting, the elastic member 160 may supply a force to restore the overall length of the foot support 7 back to its adjusted length. Thus, the elastic member 160 may help to keep the foot support 7 secured onto the shoe of the user.

Conversely, when the distance between the first support 100 and the second support 200 is less than a predetermined distance, the elastic member 160 may be compressed, and may apply a force such that the distance between the first support 100 and the second support 200 may be increased. The user may adjust the button dial 400 so that the elastic member 160 is compressed in accordance with a small shoe size, and the elastic member 160 may supply an elastic force in a direction in which the entire length of the foot support 7 is increased. Details on how the button dial 400 can control an expansion and compression of the elastic member 160 will be described later with reference to FIG. 18. When the user unbuckles the strap 300 from the button dial 400, the entire length of the foot support 7 may be increased due to a release of the elastic member 160, whereby the user may easily separate the shoe from the foot support 7.

In an alternative embodiment, the elastic member 160 may supply an elastic force only capable of reducing the distance between the first support 100 and the second support 200. In this case, the size of the foot support 7 may be kept to a minimum when it is not in use. In such a case, the button dial 400 may not be able to control the elastic member 160.

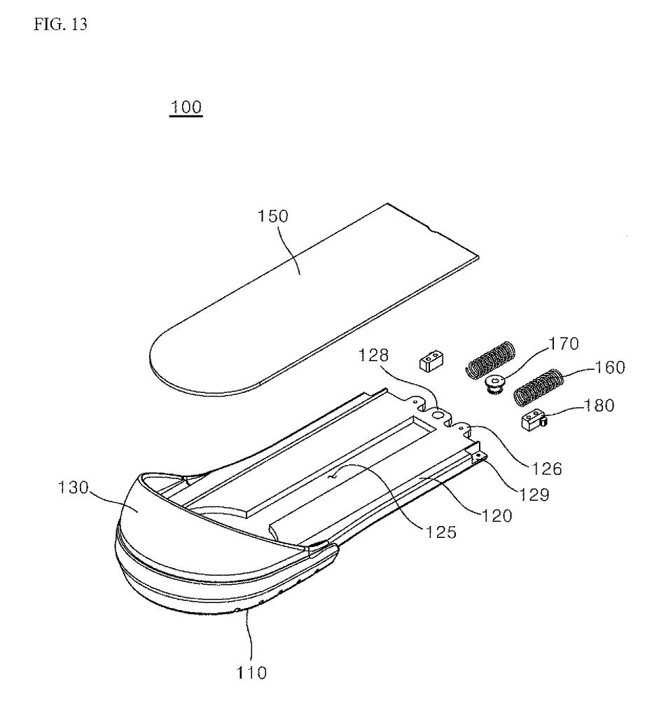

The midsole 120 may be provided with a first stepped groove or a midsole groove 125 that is formed concavely inward from an upper surface of the midsole 120 therein (FIG. 13). Although not explicitly shown in the drawings, a first pressure sensor capable of measuring a front load of the user who wears the foot support 7 may be provided in the midsole groove 125.

Similarly, the rear housing 220 may be provided with a second stepped groove or a rear recess 225 that is formed concavely inward from an upper surface of the rear housing 220 therein. Although not explicitly shown in the drawings, a second pressure sensor capable of measuring a rear load of the user who wears the foot support 7 may be provided in the rear recess 225. The midsole groove 125 and the rear recess 225 may be provided on different planes. For example, the midsole groove 125 may be provided further from the ground than the rear recess 225. In other exemplary embodiments, positions of the midsole groove 125 and the rear recess 225 may be modified.

The first pressure sensor (not shown) and the second pressure sensor (not shown), which are respectively provided in the midsole groove 125 and the rear recess 225, may be used to measure a movement of the user who wears the exoskeleton A. Data measured by the first pressure sensor (not shown) and the second pressure sensor (not shown) may be transferred to the main controller or the subcontroller via a data cable or wire housed within a shaft stabilizer 550 that intersects a longitudinal direction of the ankle support 500. The main controller or the subcontroller may calculate a magnitude of an assistive force supplied to the user who wears the exoskeleton A on the basis of the received data, and control an operation of the driving means in the actuated hip joint 3 or the actuated joint 6b.

Hereinafter, each constituent element of the foot support 7 will be described in detail with reference to the exploded perspective views of FIGS. 12-16 of the foot support 7. Referring to FIGS. 12 and 13, the first support 100 may include the first outer sole 110 that is in contact with the ground, the midsole 120 that is partially inserted into the second support 200, and the toe cover 130 that extends upward from an end of the midsole 120 and/or the first outer sole 110. The toe cover 130 may have a shape to cover a portion of the front of the shoe so as to prevent the front of the shoe from separating from the foot support 7.

The first support 100 may include an upper layer 150 described layer that covers an upper surface of the midsole 120. A first protruding member or protrusion 126 coupled to the elastic member 160, a second protruding member or protrusion 128 coupled to a wire roller or winder 170, and a stopper slot or recess 129 coupled to a stopper 180 formed at an end of the first support 100.

As previously described, the first outer sole 110 may be formed to protrude downward from the midsole 120, and various patterns capable of enhancing a frictional force with the ground may be formed on a bottom surface of the first outer sole 110 that is in contact with the ground. The midsole 120 may have a long and flat shape to support the front of the user's shoe. A portion of the midsole 120 may be inserted into the inner space 235 of the second support 200 to perform a sliding operation. The midsole 120 may be provided with the midsole groove 125 formed concavely inward. The midsole groove 125 may include a linear stepped portion or linear groove having a predetermined width and formed across a longitudinal direction of the midsole 120, and a circular stepped portion or circular recess formed at an end of the linear groove. The midsole groove 125 is not limited to the shape shown in the drawings, and may be configured in various shapes.

The first pressure sensor capable of measuring the front load or weight of the user may be provided in the circular recess of the midsole groove 125. A line or wire and additional circuits that transmit data measured by the first pressure sensor may be provided in the linear groove of the midsole groove 125. The midsole groove 125 may be inserted into the second support 200 so that the wire connected to the first pressure sensor is connected to an inner side of the second support 200. The wire may further connect to the second pressure sensor provided in the rear recess 225. Alternatively, there may be a separate wire connecting the second pressure sensor in the rear recess 225 to the main controller and/or subcontroller. The wire(s) connected to the first and second pressure sensors are not shown, and may be different from the wire W connected to the strap 300.

The upper layer 150 may be provided on the midsole 120 so as to cover an upper surface of the midsole groove 125. The upper layer 150 may support a bottom surface of the user's shoe. The upper surface 150 may include a layer made of a soft, elastic, or shock absorbing material to provide comfort to the user and to absorb any impact to serve as an inner sole. The upper layer 150 may be made of a material having a large coefficient of friction to prevent the shoe from slipping, and/or a shock-absorbent material so as to absorb an impact received from the ground during walking, bending, or lifting.

The elastic member 160 may be coupled to the first protrusion 126 formed at the first end of the midsole 120. A plurality of elastic members 160 may be coupled to the midsole 120, and a number of first protrusions 126 may be formed to correspond to a number of the elastic members 160. The number of the elastic members 160 coupled to the midsole 120 is not limited to the two shown in FIG. 13, and may be changed, as necessary.

A wire roller or winder 170 may be coupled to the second protrusion 128. The second protrusion 128 may be installed at a center of the first end of the midsole 120. The wire roller 170 may be provided with a wire W that is connected to the button dial 400 through the strap 300. As a length of the wire W is adjusted by an operation of the button dial 400, a distance between the first support 100 and the second support 200 may also be adjusted.

There may be a plurality of second protrusions 128 and a plurality of wire rollers 170, and a number of the second protrusions 128 coupled to the midsole 120 may correspond to a number of the wire rollers 170 coupled to the second protrusions 128. The number of second protrusions 128 and wire rollers 170 are therefore not limited to that as shown in FIG. 13, and may be changed, as necessary.

A stopper 180 may be coupled to a stopper slot or recess 129. The stopper 180 may be formed in pair to correspond to a pair of stopper recesses 129 formed on opposite sides of the first end of the midsole 120. The stopper recess 129 may be a cut portion of the midsole 120.

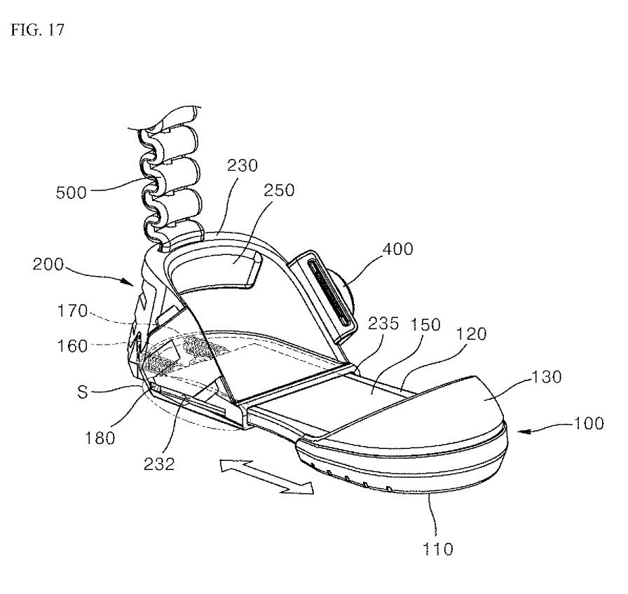

When the stopper 180 is coupled to the stopper recess 129, a portion of the stopper 180 may protrude from a side surface of the midsole 120. A protruding portion of the stopper 180 may be coupled to a stopper slot or stopper guide 232 formed on an inner side of the second support 200 to prevent the first support 100 from separating from the second support 200. The stopper guide 232 may also be referred to as a stopper hole. Alternatively, the stopper guide 232 may be a groove. The stopper guide 232 may restrict a maximum distance between the first and second supports 100 and 200, and thus restrict a maximum length of the foot support 7. A detailed description thereof will be described later in detail with reference to FIG. 17.

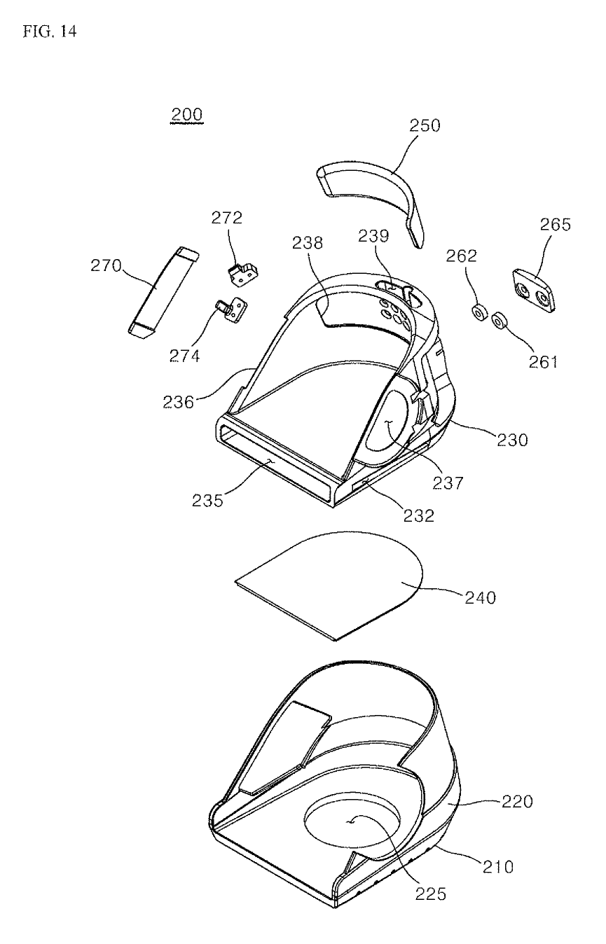

Referring to FIGS. 11 and 14, the second support 200 may include the rear housing 220 and the rear body 230 coupled to an inner surface of the rear housing 220. The rear housing 220 may form an outer surface of the second support 200, and a lower surface of the rear housing 220 may be provided or formed with the second outer sole 210 that is in contact with the ground.

The second outer sole 210 may be provided on the same plane as the first outer sole 110 of the first support 100 described above. As previously described, various patterns capable of enhancing a frictional force with the ground may be formed on a bottom surface of the second outer sole 210 that is in contact with the ground.

The rear housing 220 may be provided with the rear recess 225 that is formed concavely inward. The second pressure sensor capable of measuring a rear load or weight of the user may be provided in the rear recess 225. Although not explicitly shown in the drawings, a line or wire and additional circuits to transmit data measured by the second pressure sensor may be provided between the rear housing 220 and the rear body 230.

A rear or supporting plate 240 that covers an upper surface of the rear housing 220 may be provided between the rear housing 220 and the midsole 120, i.e., the rear plate 240 may be provided above the rear housing 220 and below the midsole 120. Alternatively, the rear plate 240 may be provided between the rear housing 220 and the rear body 230. The rear plate 240 may be made of a shock-absorbent material to reduce an impact when the user walks, bends, or lifts. The rear plate 240 may be optional, and in another exemplary embodiment, the rear plate 240 may be omitted.

The rear body 230 may be coupled to an inner surface of the rear housing 220. The inner space 235 in which the midsole 120 is inserted may be provided in or adjacent to the rear body 230. The inner space 235 may be formed within the rear body 230. Alternatively, the inner space 235 may be provided between the rear housing 220 and the rear body 230. As another alternative embodiment, the inner space 235 may be provided in the rear shaft housing 220. Hereinafter, a configuration of the inner space 235 being formed in the rear body 230 will be described as an example.

The stopper guide 232 may open a portion of a side surface of the rear body 230, and may be formed at outer sides of the inner space 235. The stopper guide 232 may accommodate the stopper 180 that is provided in the midsole 120 after the midsole 120 is inserted into the inner space 235. The stopper 180 that protrudes from a side surface of the midsole 120 may be coupled to the stopper guide 232, and may move along the stopper guide 232.

Accordingly, the stopper guide 232 may restrict a predetermined range of motion of the first support 100 in the second support 200, thereby preventing the first support 100 from being separated from the second support 200. For reference, the stopper guide 232 may be formed on opposite sides of the rear body 230, and rear housing 220 but embodiments disclosed herein are not limited thereto. For example, the stopper guide 232 may be formed only on one side of the rear body 230. In this embodiment, the stopper 180 may be provided on only one surface of the midsole 120 corresponding to the stopper guide 232.

Referring to FIG. 14, a first side of the rear body 230 and/or rear housing 220 may be provided with a strap groove or recess 236 into which the strap 300 is inserted and a buckle stopper or strap cover 270 that partially or fully covers the strap groove 236 to prevent the strap 300 from being completely detached from the second support 200.

The buckle stopper 270 may be installed on the rear body 230 at a predetermined height, and may not allow the wire guide 350 coupled to the strap 300 to pass past the buckle stopper 270 to the strap groove 236. In addition, wire guides 272 and 274 may be installed on the first side of the rear body 230 to guide a movement of the wire W connected to the button dial 400. Positions of the wire guides 272 and 274 may be changed or omitted depending on the movement or line of motion of the wire W.

A button dial groove 237 to which the button dial 400 is coupled may be formed on a second side of the rear body 230. Alternatively, the button dial groove 237 may be a ledge. The button dial groove 237 may be optional.

Wire rollers 261 and 262 to guide a path of the wire W and a wire roller cover 265 to fix the wire rollers 261 and 262 to a rear surface of the rear body 230 and/or rear housing 220 may be installed on the rear surface of the rear body 230 and/or rear housing 220. Positions of the wire rollers 261 and 262 and the wire roller cover 265 may be changed or omitted depending on the line of motion of the wire W.

A heel cushion groove 238 in which the heel cushion 250 is provided may be formed on an inner surface of the rear body 230. The heel cushion groove 238 may be formed on an upper side of the inner surface of the rear body 230. The heel cushion 250 may protrude outward from the inner surface of the second support 200, and may be provided on an upper or top side of the second support 200. The heel cushion 250 may support an upper heel of the user (e.g., an upper Achilles tendon).

The heel cushion 250 may be made of a different material from the rear body 230. For example, the heel cushion 250 may be made of a material having elasticity or a resilience. Accordingly, when the user who wears the wearable assistive device walks, bends, or lifts, the heel cushion 250 may alleviate an impact transferred to the user's heel and improve comfort. For reference, the heel cushion 250 may be configured in various shapes and positions, or may be omitted.

An ankle groove or opening 239 to fix the ankle support 500 may be formed on a center of the upper surface of the rear body 230. Alternatively, the ankle groove 239 may be omitted. In this case, the ankle support 500 may be coupled between the rear housing 220 and the rear body 230 or to an outer surface of the second support 200.

Referring to FIGS. 12 and 15, the strap 300 may include the band 310, the buckle tongue 320 that is installed to a first end of the band 310 to fasten to the button dial 400, the buckle base 340 that covers where the buckle tongue 320 connects to the band 310, and the wire guide 350 that is installed at a second end of the band 310 to be coupled to the wire W.

The band 310 may include an elastic or flexible material. For example, the band 310 may be manufactured by weaving a number of elastic or flexible fibers. However, this is merely one example, and the band 310 may be made of materials having various elasticity or flexibility.

The buckle tongue 320 may be coupled to a latch hook (442 of FIG. 16) of the button dial 400. The buckle tongue 320 may have a slit or latch ring that couples to or hooks onto the latch hook 442. In addition, the buckle tongue 320 may have at least one coupling groove or connection hole that is couplable to protrusions on clip holders 332 and 334. The at least one coupling groove may be provided on a side of the buckle tongue 320 opposite to a side in which the slit is formed.

The clip holders 332 and 334 may fix the buckle tongue 320 to the band 310. The clip holders 332 and 334 may have protrusions and/or connection holes that pass through or couple to the first side 312 of the band 310 and the coupling grooves or connection holes of the buckle tongue 320. In the example shown in FIG. 15, binding clip holder 332 may have protrusions or bolts which pass through connection holes of clip holder 334 and buckle tongue 320. The first side 312 of the band 310 may have an opening through which the protrusions of clip holder 332 pass.

The buckle base 340 may include first and second covers 342 and 344. The buckle base 340 may be assembled to cover outer surfaces of the clip holders 332 and 334. When the strap 300 is coupled to the button dial 400, the buckle base 340 may be brought into contact with a dial cover or housing (430 of FIG. 16) of the button dial 400.

The wire guide 350 may be coupled to the second end of the band 310. The wire guide 350 may be coupled to at least one connection hole 314 provided at the second end of the band 310. In the example shown in FIG. 15, the wire guide 350 may have a base having connection holes that correspond to the connection holes 314 of the band 310. Coupling members such as screws, bolts, shafts, pins, etc. may pass through the connection holes 314 of the band and the connection holes of the wire guide 350.

The wire guide 350 may have a protrusion or stopper that extends from the base of the wire guide 350 having the connection holes. The stopper of the wire guide 350 may prevent the strap 300 from being completely detached from the rear body 230. The buckle stopper 270 of FIG. 14 may be installed on the rear body 230 so that the stopper of the wire guide 350 may not pass through an opening or space created between the buckle stopper 270 and the strap groove 236. As a result, the wire guide 350 may be maintained on the inner side of the second support 200.

Referring to FIGS. 12 and 16, the button dial 400 may include a button or dial 420, a dial cover 430, a winding or driving assembly 440, and a dial base 410 (FIG. 12). The dial base 410 may be provided with a receiving space or opening 450 into which the buckle tongue 320 may be partially inserted. The dial base 410 may be coupled to the button dial groove 237 provided on the second side of the rear body 230. The receiving space 450 may also be referred to as a buckle hole or coupling groove.

The winding assembly 440 may fix to the buckle tongue 320 so that the strap 300 is coupled to the button dial 400, and may wind the wire W to adjust the length of the wire W. The winding assembly 440 may include a dial housing or base frame 441, a latch hook 442, a latch holder 443, a wire winding body or case 445, a wire winding dispenser 447, and a winding cover or button body 449. The wire winding dispenser 447 may be one of a reel, bobbin, and spindle. For convenience of description, the wire winding dispenser 447 will be referred to as a reel, and the case 445 will be referred to as a reel case.

The dial housing 441 may be provided on the dial base 410. The latch hook 442 may be rotatably coupled to the dial housing 441 by the latch holder 443. The latch holder 443 may have a block shape that secures protrusions of the latch hook 442 onto latch holder grooves provided on an outer surface of the dial housing 441.

The latch hook 442 may be coupled to the buckle tongue 320 to fix the strap 300 to the button dial 400, or may detach from the buckle tongue 320 to separate the strap 300 from the button dial 400 depending on an angle. The latch hook 442 may have a latch stick that inserts into the slit of the buckle tongue 320.

The reel case 445 may have the reel 447 mounted therein, and an outer surface of the reel case 445 may be covered with the winding cover 449. The reel case 445 and the winding cover 449 may form an inner space, and the reel 447 may be provided in the inner space. The wire W may be wound on a side surface or inner spool of the reel 447. The reel 447 may have a hole that penetrates through the inner spool, and the hole may fit onto a projection that protrudes from inside of the reel case 445.

Although not explicitly shown in the drawings, the reel 447 may be provided with an elastic body or a gear such as a spring therein. As a result, the elastic body may apply a restoring force to rotate the reel 447 in a predetermined direction. Further, the winding cover 449 may be coupled to an outer surface of the reel 447. Therefore, when the winding cover 449 rotates, the reel 447 may also rotate.

The reel case 445 may be mounted on the dial housing 441. The winding cover 449 may be coupled to the dial base 410 to cover outer and side surfaces of the dial housing 441 so that the dial housing 441 and the latch hook 442 are not exposed.

The dial 420 may be coupled to the winding cover 449. The dial 420 may be exposed to an outside to be rotated by the user, and a force applied to the dial 420 may be transferred to the reel 447. The user may thus rotate the reel 447 by operating the dial 420 to adjust or wind the wire W.

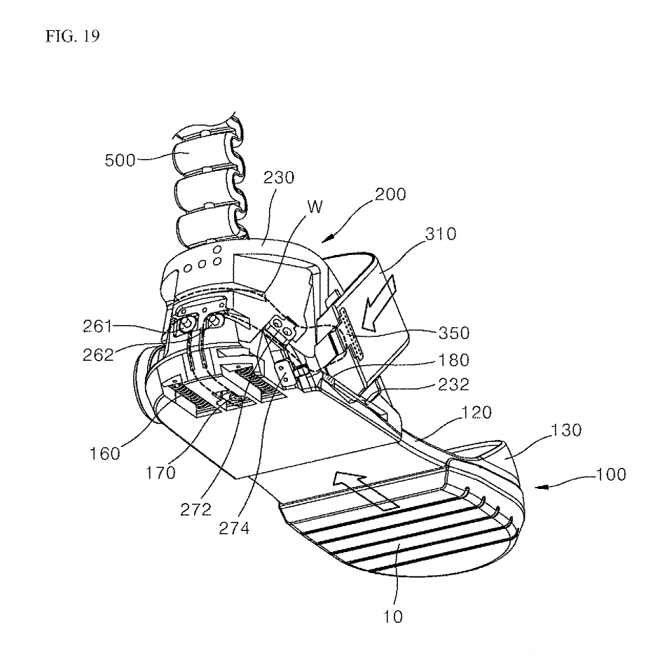

The dial 420 may include a dial cap 421, a dial frame or ring 423, and a dial body 425. The dial cap 421 and the dial frame 423 may be omitted or formed integrally with the dial body 425 to form the appearance of the dial 420. The dial frame 423 may be referred to as a dial decoration. A relationship between the wire W and the strap 300 will be explained later with reference to FIGS. 18 and 19.

FIG. 17 is a view explaining a movement between the first support and the second support of the foot support of FIG. 5. Referring to FIG. 17, the first support 100 provided in the foot support 7 may be inserted into the second support 200 to be slideably coupled. The midsole 120 of the first support 100 may be inserted into the inner space 235 of the second support 200. The stopper 180 that protrudes from a side surface of the midsole 120 may be coupled to the stopper guide 232 formed on a side surface of the rear body 230. The stopper guide 232 may be connected to or communicate with the inner space 235 formed on an inner side of the rear body 230.

The stopper guide 232 may be coupled to the stopper 180 after the midsole 120 inserted into the inner space 235. A protrusion of the stopper 180 that protrudes from the side surface of the midsole 120 may be guided along the stopper guide 232 in a one dimensional or liner movement. The second support 200 may therefore guide a sliding of the first support 100 so that the first support 100 is not detached from the second support 200, Accordingly, the stopper guide 232 may restrict a movement of the first support 100 such that it slides within a predetermined range of the second support 200, thereby preventing the first support 100 from being separated from the second support 200.

FIGS. 18 and 19 are views for explaining operations of the strap and the button dial provided in the foot support of FIG. 5. In order to explain a path of the wire W and a driving or winding principle of the foot support 7, the rear housing 220 that covers the outer surface of the second support 200 and some other elements are omitted in FIGS. 18 and 19. Referring to FIGS. 18 and 19, positions of the strap 300 and the first support 100 may be adjusted by the wire W connected to the button dial 400.

The button dial 400 may include the dial 420 rotated by the user, the winding assembly 440 to adjust the length of the wire W in response to a rotation of the dial 420, and the dial base 410 having the receiving space 450. The dial base 410 may be fixed to the button dial groove 237 (FIG. 14) formed on the first side of the rear body 230, The buckle tongue 320 of the strap 300 may be inserted into the receiving space 450.

The buckle tongue 320 of the strap 300 may be coupled to or separated from the button dial 400 by a latch hook 442 included in the winding assembly 440. The winding assembly 440 may be installed on the dial base 410. The dial 420 may be coupled to an outer surface of the winding assembly 440 to control an operation of the winding assembly 440.

The winding assembly 440 may adjust the length of the wire W by winding or unwinding the wire Win response to the rotation of the dial 420. When the dial 420 is rotated in a winding direction or when the strap 300 is unbuckled, the reel 447 may wind the wire W, thereby tightening the strap 300. When the dial 420 is rotated in an unwinding direction or when the strap 300 is withdrawn, the reel 447 may unwind the wire W, thereby loosening the strap 300.

The dial 420 may change a position of the latch hook 442 included in the winding assembly 440. As the dial 420 is pushed inward or outward, a latch stick of the latch hook 442 may be pressed or pulled into or out of a slit of the buckle tongue 320 to buckle or unbuckle the strap 300 to or from the button dial 400. For example, the user may push the dial 420 inward to place the dial 420 in a pressed state. When the dial 420 is rotated in a pressed state, the wire W may be wound. In this case, the strap 300 may remain fixed to the button dial 400. When the wire W is wound, a “winding amount” of the wire W may increase.

On the other hand, when the button type dial 420 is pulled outward to a pulled state while the strap 300 is fixed to the button dial 400, the position of the latch hook 442 may move to decouple from the slit of the buckle tongue 320, and the strap 300 may be unfastened from the button dial 400. For reference, the method of operating the dial 420 may be modified in various ways.

Both ends of the wire W may be connected to the winding assembly 440 of the button dial 400. An intermediate path of the wire W may be defined as a path of the wire to around the wire rollers 261 and 262 and through the wire guides 272 and 274 provided on the outer surface of the rear body 230. The wire rollers 261 and 262 may be installed on the rear surface of the rear body 230, and the wire roller cover 265 may fix the wire rollers 261 and 262 to the rear surface of the rear body 230 and/or the rear housing 220, and the wire guides 272 and 274 may be installed on a first side of the rear body 230.

For reference, the wire rollers 261 and 262 and the wire guides 272 and 274 may be repositioned in response to the path of the wire W, or may be omitted. Further, the number of wire rollers 261 and 262 and/or wire guides 272 and 274 may vary.

The wire guide 350 of the strap 300 may be positioned to be a part of the intermediate path of the wire W. Similarly, the wire roller 170 protruding from the midsole 120 may also be positioned to be a part of the intermediate path of the wire W. The wire W may pass around the wire roller 170 and through the wire guide 350.

When the dial 420 of the button dial 400 is rotated to wind the wire W, the tension of the wire W may be increased. As the tension of the wire W is increased, an end of the strap 300 having the wire guide 350 may move toward an inner side of the second support 200, and the first support 100 may move toward the second support 200.

As the length of the wire W is reduced by winding the wire W, an exposed the length of the strap 300 may be reduced, thereby tightening the strap and securing an upper surface of the shoe. Further, overall length of the foot support 7 may also be reduced as the midsole 120 is further inserted into the securing space 235 of second support 200. Accordingly, midsole 120 and the rear body 230 also may tighten the front and rear of the shoe more firmly, as the distance between the first and second supports 100 and 200 is reduced.

The wire W may connect the strap 300, via the wire guide 350, and the first support 100, via the wire roller 170 of the midsole 120, so as to have one path. When a distance between the first support 100 and the second support 200 is minimized based on a size of the shoe in the foot support 7, only the length of the strap 300 may be adjusted, i.e., tightness and firmness of the soot support 7 is maximized in the longitudinal direction, a winding of the button dial 400 may only tighten the strap 300.

Conversely, when of the strap 300 tightly covers the top of the foot of the shoe such that an exposed length of the strap 300 is minimized, only the the first support 100 and the second support 200 may be adjusted. In this case, the wire W may pass through the wife guide 350 without moving or pulling the bond 310 into the receiving space 235. Thus, the user may adjust the foot support 7 in accordance with the entire length of the user's shoe and the height of the shoe by rotating the button dial 400, and the user's shoe may be firmly fixed to the foot support 7.

The wire W may connect the strap 300 and the first support 100 via the wire guide 350 and the wire roller 170 from one line of motion or path of the wire W. When the user pulls the button type dial 420 to unbuckle the strap 300 from the button dial 400, the length of the wire W may be automatically increased as the wire is unwound. When the wire is unwound, the “winding amount” may be decreased.

The first support 100 may be moved in a direction away from the second support 200 by the elastic force of the elastic member 160, and the exposed length of the strap 300 may be increased.

The user may easily take the foot support 7 off of his shoe through a one-touch operation (for example, pushing or pulling operation) of the button dial 400.

In more detail, the wire W may have first and second ends, both connected to the winding assembly 440. For example, the first end of the wire W may be fixed to the reel 447, and the second end of the wire W may be fixed to an interior surface of the reel case 445. Alternatively, the wire W may be fixed to an outer circumferential surface of the reel case 445 to minimize a tangling of the wire W. Thus, as the reel 447 rotates in the reel case 445, the wire W may be wound around the reel 447.

While the first end of the wire W is fixed to the reel 447, the second end of the wire W may follow the intermediate path. The wire W may extend from the reel 447 and be guided out of the button dial 400 under the dial cover 430 and over the dial housing 441. An outer surface of the rear body 230 and/or the rear housing 220 may have guide grooves through which the wire W may be placed in order to keep the wire W taut and minimize tangling as it extends around the wire roller 261, then around the wire roller 170, and then around the wire roller 262. The wire W may then pass through the wire guide 274, the wire guide 350, and then the wire guide 272. From the wire guide 272, the wire W may then extend across the rear body 230, and the second end of the wire W may then fix to the reel case 445.

In an alternative embodiment, the wire W may take a reverse path; i.e., the wire W may extend from the reel 447 across the rear body 230 to the wire guide 272, to wire guide 350, and then to wire guide 274, and then may pass around the wire rollers 262, 170, and then 261 before the second end of the wire W is fixed to the reel case 445. Embodiments disclosed herein are not limited to the above-described wire path, and may vary so long as the wire W couples to the winding assembly 440, the midsole 120 (by, for example, the wire roller 170), and the band 310 (by, for example, the wire guide 350). Thus, as the wire W tightens as it is wound around the reel 447, the band 310 may be pulled into the strap groove 236 of the second support 200, and the midsole 120 may slide into the receiving space 235 of the second support 200.

When a size of the user's shoe prevents the band 310 from being further inserted into the strap groove 236 of the second support 200, the band 310 may be more taut than the wire W. Thus, when the user continues to rotate the button dial 400 to wind the wire W, the wire W may pass through the wire guide 350, while the wire guide 350 remains stationary and is not pulled downward into the receiving space strap groove 236. Thus, the wire W may only adjust a sliding of the midsole 120 by pulling onto the wire roller 170.

When a size of the user's shoe prevents the midsole 120 from further sliding into the receiving space 235, the wire may not pull onto the wire roller 170 to drag the midsole 120 into the receiving space 235, and may instead move around the wire roller 170 while the wire roller 170 remains stationary. Thus, the wire W may only adjust a tightening of the band 310 by pulling the band 310 into the strap groove 236.

When the user pulls the button dial 400 outward to unbuckle the strap, the wire W may loosen. A restoring force of the elastic member (not shown) in the winding assembly 440 may unwind the wire W from the reel 447, and also an unfastening of the band 310 may also reduce the tension in the wire W. When the wire W is unwound from the reel 447, the wire W may become loose enough such that the band 310 may be pulled back out of the strap groove 236. A tension of the wire W may therefore no longer be keeping the band 310 tight or the midsole 120 inserted into the second support 200 at its current position. The restoring force of the elastic member 160 may thus overcome the tension force in the wire W around the wire roller 170, pushing the midsole 120 out of the second support 200 via the receiving space 235. As a result, the time required for the user to wear the exoskeleton A may be reduced, improving convenience.

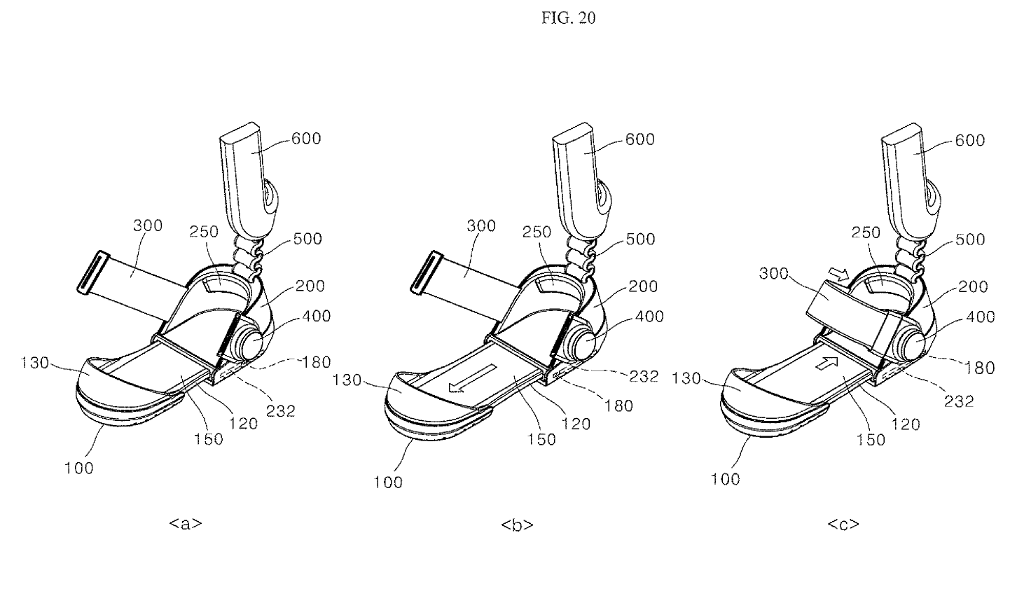

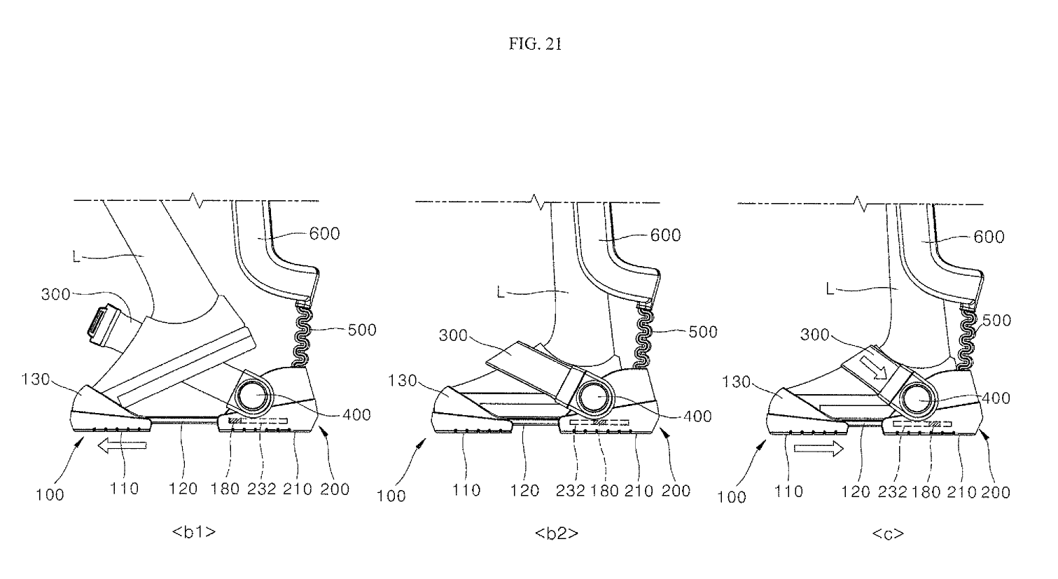

FIGS. 20 and 21 are views explaining an operation of the foot support of FIG. 5. Referring to FIG. 20, view (a) shows a state of the foot support 7 before the user's shoe is inserted into the foot support 7.

In this state, the first support 100 and the second support 200 may be spaced apart from each other by a predetermined distance by the elastic member 160. The stopper 180 may have a default or reference position in an intermediate region of the stopper guide 232. The default position of the stopper 180 may represent a position of the stopper 180 when no external force, such as a forward force while walking, bending, or lifting, or a tension force of the wire W, is applied to the first support 100.

The default position of the stopper 180 may be adjusted based on a shoe size of a main user, and may be modified in various ways. For example, the default position of the stopper 180 may be set as a point at which the entire length of the foot support 7 is at a minimum (i.e., a point at which the first support 100 is positioned closest to the second support 200). A length of the elastic members 160 may determine the default position of the stopper.

View (b) of FIG. 20 shows a state in which the entire length of the foot support 7 is increased by increasing a distance between the first support 100 and the second support 200.

Referring to FIG. 21, view (b1) shows that the user may exert a forward force when the shoe is placed on the first support 100 so as to push the shoe into the foot support 7. In this case, the distance between the first support 100 and the second support 200 may be increased. The first support 100 may move forward 7 within a movable range in which the stopper 180 moves in the stopper guide 232. The elastic member 160 may be expanded.

View (b2) shows the strap 300 fastened to the button dial 400 so as to extend across the upper surface of the user's shoe. In this case, the first support 100 may be retracted back toward the second support 200 by a restoring force of the elastic member 160 as the shoe is correctly positioned in the foot support 7 and no longer exerts a forward force. Since the elastic member 160 is fixed to the inner surface of the first support 100 and engaged with an inner surface of the second support 200, the elastic member 160 may supply the restoring force in a direction toward the second support 200, pulling the first support 100 closer to the second support 200.

The stopper 180 may be moved toward the default position in the stopper guide 232, but may not necessarily reach the default position. Thus, the elastic member 160 may still be somewhat expanded depending on how large the user's shoe is.

Views (c) of FIG. 20 and (c) of FIG. 21 show that the length of the strap 300 and the entire length of the foot support 7 may be adjusted by rotating the button dial 400. As described above, by rotating the button dial 400, the user may adjust the size of the foot support 7 by adjusting the distance between the first and second supports 100 and 200 in accordance with a shoe length of the user and adjust the length of the strap 300 in accordance with a shoe width of the user.

The first support 100 may move toward the second support 200 within a range of motion of the stopper 180 within the stopper guide 232. The strap 300 and the first support 100 may be connected by the wire W via the wire guide 350 and the wire roller 170. Accordingly, when the position of the first support 100 is as close as possible to the second support 200 in view of the shoe size, the length of the strap 300 may be adjusted independently from an adjustment of the distance between the first and second supports 100 and 200. Also, when an exposed length of the strap 300 is as short as possible in view of the shoe width, the distance between the first support 100 and the second support may be adjusted independently from an adjustment of the strap 300. As a result, the user may adjust the foot support 7 in accordance with the entire length and height or width of the shoe, and the user's shoe may be firmly fixed to the foot support 7.

The foot support 7 of the exoskeleton A according to embodiments disclosed herein may firmly secure the user's shoe to the foot support 7. Also, the foot support 7 may improve safety and prevent a tripping accident that may occur due to a separation of the shoe while walking, bending, or lifting, thereby improving reliability and safety of the operation of the exoskeleton A.

Embodiments disclosed herein may provide a wearable assistive device including a foot unit or support whose length is adjusted in accordance with a shoe size of a user so that the user wears the wearable assistive device with shoes on. The wearable assistive device may be a power assisting robot or exoskeleton.

Embodiments disclosed herein may provide a wearable assistive device including a foot unit or support capable of securing various forms of shoes regardless of a type or a size. The foot support may secure an upper surface of a shoe using a binding portion or strap and may adjust a length of the strap through a rotary dial or button dial to facilitate fastening and unfastening of the shoe. The foot support may be capable of adjusting an entire length of the foot support or a length of the strap in a one-touch manner.

Embodiments disclosed herein are not limited to the above-mentioned objects, and the other objects and the advantages of this disclosure which are not mentioned can be understood by the following description, and more clearly understood by the exemplary embodiments of this disclosure. It will be also readily seen that the objects and the advantages of this disclosure may be realized by means indicated in the patent claims and a combination thereof.

The foot support of the wearable assistive device according to embodiments disclosed herein may include a front shaft fixing portion or first support and a rear shaft fixing portion or second support that are slidingly coupled to each other, thereby adjusting the length of the foot support in accordance with the shoe size of the user. Further, the first support may cover a portion of a front shaft of the user's shoe, and the second support may support a rear shaft of the shoe, thereby fixing various forms of shoes regardless of the type and size of the shoe.

Furthermore, the foot support may include a binding portion or strap that covers an upper surface of the shoe and a button dial that adjusts a length of the strap exposed to the outside, thereby facilitating fixing and detachment of the shoe. In addition, the button dial may adjust a winding amount of a wire in a one-touch manner to adjust the entire length of the foot support or the length of the strap, thereby improving an operation convenience of the user.

The foot support may be adjustable in length in accordance with the shoe size of the user by means of the first support and the second support that are slidingly coupled to each other. The length of the foot support may be extended in accordance with the shoe size of the user, reducing an inconvenience of adjusting a size of the foot support for a particular user. Users with different shoe sizes may use the same foot support, thereby increasing the versatility of the wearable assistive device. By increasing in the versatility of the wearable assistive device, it may be possible to mass-produce wearable assistive device having a same or substantially similar structure, thereby reducing manufacturing cost and improving the operation efficiency of the wearable assistive device.

The first support may cover a portion of the front shaft of the user's shoe and the second support may support the rear shaft of the shoe. The first support 100 and the second support 200 may be separated from each other, thereby able to secure various forms of shoes regardless of a type and a size of the shoe. Shoes having various shapes and sizes may be secured by using a structure in which the front and rear shafts of the shoe are attached to the foot support, and thus various users may use the foot support.

Further, the user may easily secure the front and rear shafts of the shoe to the foot support by pushing the first support forward, thereby reducing the time required for wearing the wearable assistive device and increasing the comfort of the user. The strap may cover the upper surface of the shoe and the button dial may adjust the length of the strap exposed to the outside, thereby firmly securing the user's shoe to the foot support. The strap may be fastened to or separated from the button dial in a one-touch manner or the length thereof may be adjusted, thereby reducing the time required for wearing the wearable assistive device and increasing the convenience of the user. Further, the strap may prevent the shoe from being easily detached from the foot support even when the user wears the wearable assistive device and walks, bends, or lifts. As a result, it is possible to prevent a safety accident that may occur due to the separation of the shoe while walking, bending, or lifting, thereby improving the reliability and safety of the operation of the power assisting robot.

The foot support may include a button dial portion or button dial that adjusts a winding amount of the wire in a one-touch manner to adjust the entire length of the foot support or the length of the strap, thereby improving the operation convenience of the user. Furthermore, the button dial may unfasten the strap in a one-touch manner or adjust the entire lengths of the strap and the foot support, thereby firmly securing the shoe to the foot support. As a result, it may be possible to reduce the time required for donning or preparing to don the wearable assistive device and increase the user's convenience.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present invention.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A wearable assistive device, comprising:
a main frame configured to support a waist or a pelvis;
a leg assembly that extends from an end of the main frame; and
a foot support provided at an end of the leg assembly,
wherein the foot support includes:
a first support that partially covers a front of a foot or shoe; and
a second support that has an inner space in which the first support is inserted, the second support supporting a rear of the foot or shoe.

2. The wearable assistive device of claim 1, further comprising an elastic member that is provided in the inner space of the second support to provide an elastic force between the first and second supports.

3. The wearable assistive device of claim 2, wherein the elastic member has a first end fixed to an inner side of the first support and a second end that pushes against an inner space of the second support.

4. The wearable assistive device of claim 1, wherein the first support includes:
a first outer sole that is in contact with the ground;
a midsole that is spaced apart from the ground and inserted into the inner space of the second support; and
a toe cap that extends upward from the midsole.

5. The wearable assistive device of claim 4, wherein the midsole includes a first stepped groove that is formed concavely inward from an upper surface of the midsole, and also includes a cover that covers an upper surface of the first stepped groove.

6. The wearable assistive device of claim 5, wherein the second support includes:
a rear housing that is provided with a second outer sole that is in contact with the ground and a second stepped groove that is formed concavely inward; and
a rear body coupled to an inner surface of the rear housing to cover an upper surface of the second stepped groove, the rear body housing the inner space therein.

7. The wearable assistive device of claim 6, wherein the first stepped groove and the second stepped groove are provided on different planes.

8. The wearable assistive device of claim 4, wherein the first support further includes a stopper that protrudes from a side surface of the midsole, and the second support includes a stopper guide in which the stopper moves.

9. The wearable assistive device of claim 8, wherein a range of motion of the first support is equal to a length of the stopper guide.

10. The wearable assistive device of claim 1, wherein the second support includes a heel cushion that protrudes from an inner surface of the second support to contact a rear of the shoe or foot.

11. A foot support, comprising:
a supporting structure that supports a shoe,
a strap that extends from a first side of the supporting structure to extend across an upper surface of the shoe to a second side of the supporting structure, and
a button dial provided at the second side of the supporting structure and selectively fastened to the strap, wherein the button dial adjusts a length of the strap outside of the supporting structure when the strap is fastened to the button dial.

12. The foot support of claim 11, wherein the supporting structure includes:
a first support to partially cover a front of the shoe; and
a second support in which the first support is inserted, the second support supporting a rear of the shoe, wherein the button dial is configured to adjust positions of the strap and the first support by adjusting a length of a wire connected to the strap and the first support.

13. The foot support of claim 12, wherein the strap and the first support are connected by the wire, and the button dial adjusts a position of the strap or the first support by winding the wire.

14. The foot support of claim 13, wherein a distance between the first and second supports or an exposed length of the strap is reduced when the wire is wound.

15. The foot support of claim 12, wherein the second support includes:
a rear housing that forms an outer surface; and
a rear body coupled to an inner surface of the rear housing and provided with an inner space in which the first support is inserted, wherein the wire is provided between the rear housing and the rear body.

16. The foot support of claim 15, wherein the second support further includes:
at least one wire roller provided on a rear surface of the rear body to change a direction of the wire; and
a wire roller cover that fixes the at least one wire roller on a rear surface of the rear housing.

17. The foot support of claim 15, wherein the second support further includes a wire guide that is fixed on the rear body to change a direction of the wire.

18. The foot support of claim 11, wherein the button dial includes:
a latch hook that selectively fixes to a first end of the strap;
a reel on which the wire is wound; and
a dial that rotates the reel to wind or unwind the wire.

19. The foot support of claim 18, wherein, when the strap is fastened to the latch hook and the dial is pulled, the strap becomes unfastened from the latch hook and the reel winds the wire to reduce an exposed length of the strap.

20. The foot support of claim 11, wherein the strap comprises:
a band that is partially inserted into the second support;
a buckle tongue that is provided at a first end of the band to be coupled to the button dial; and
a wire guide that is provided at a second end of the band to connect to the wire.
